(19) United States
(12) Patent Application Publication
(10) Pub. No.: US 2022/0354404 A1
(43) Pub. Date: Nov. 10, 2022

(54) UROFLOW MONITORING SYSTEM AND METHOD

(71) Applicants: The Children's Hospital of Philadelphia, Philadelphia, PA (US); The Trustees of the University of Pennsylvania, Philadelphia, PA (US)

(72) Inventors: Jason Van Batavia, Philadelphia, PA (US); Stephen A. Zderic, Philadelphia, PA (US); James Weimer, Philadelphia, PA (US); Brett Garberman, Philadelphia, PA (US); Qizhi Li, Philadelphia, PA (US); Xin Liu, Philadelphia, PA (US); Igor Shamis, Philadelphia, PA (US); Yuriy M. Mirochnik, Philadelphia, PA (US); Alexander Gutsol, Philadelphia, PA (US)

(21) Appl. No.: 17/636,950

(22) PCT Filed: Aug. 19, 2020
(86) PCT No.: PCT/US2020/046938

§ 371 Date: Feb. 21, 2022

Related U.S. Application Data
(60) Provisional application No. 62/890,169, filed on Aug. 22, 2019.

(51) Int. Cl. A61B 5/20; A61B 5/00

(52) U.S. Cl. CPC A61B 5/208; A61B 5/002; A61B 5/743; A61B 5/486; A61B 2562/0247

(57) ABSTRACT

A uroflowmetry device including a funnel for funneling urine of a user, a sensor for measuring urine level in the funnel over a measurement period, a paddle wheel operable to rotate in response to urine exiting the funnel and generate a signal after each rotation during said measurement period, a transmitter for transmitting data from the uroflowmetry device to a smart device, and a processor. The processor configured to, while the user is urinating, compute a urine flowrate based on urine level in the funnel and a number of rotations of the paddle wheel over the measurement period, and transmit the urine flowrate to the smart device to provide real-time urination feedback to the user.

### CROSS-REFERENCE TO RELATED APPLICATIONS

The present application claims priority to U.S. Provisional Patent Application No. 62/890,169, filed Aug. 22, 2019, entitled “UROFLOW MONITORING SYSTEM AND METHOD” the contents of which are incorporated herein by reference in their entirety.

### FIELD

The subject matter disclosed herein relates to devices, systems and methods for providing real-time uroflowmetry feedback to a patient and their physician.

### BACKGROUND

Lower urinary tract (LUT) symptoms and dysfunction affect a significant portion of children. State-of-the-art uroflowmetry devices have been used to measure urine flow and provide biofeedback to patients. However, state-of-the-art uroflowmetry devices are crude devices used in a physician's office that typically include a funnel, a beaker and a scale connected to a computer. During a uroflowmetry session, the patient urinates into the funnel, and the urine is collected in the beaker. The weight of the beaker is monitored during this time period to determine uroflowmetry parameters such as urine flowrate and total volume voided. Electromyography (EMG) electrodes are sometimes attached to the patient's abdomen and perineum during the uroflowmetry session. For most children with LUT symptoms, a uroflowmetry study is obtained in the office as part of the initial evaluation and/or monitoring of treatment. Children with a specific condition called dysfunctional voiding often require multiple biofeedback sessions in the physician's office to correct abnormal pelvic floor activity during voiding. This process has many drawbacks, including but not limited to intensive and long sessions conducted at a physician's office every week that result in missed school for the patient. In addition, the use of uncomfortable patch EMG electrodes and a clinical setting can create an uncomfortable environment that makes it more difficult for the patient to relax and urinate.

### SUMMARY

An embodiment includes a uroflowmetry device including a funnel for funneling urine of a user, a sensor for measuring urine level in the funnel over a measurement period, a paddle wheel operable to rotate in response to urine exiting the funnel and generate a signal after each rotation during said measurement period, a transmitter for transmitting data from the uroflowmetry device to a smart device, and a processor. The processor configured to, while the user is urinating, compute a urine flowrate based on urine level in the funnel and a number of rotations of the paddle wheel over the measurement period, and transmit the urine flowrate to the smart device to provide real-time urination feedback to the user.

Another embodiment includes a smart device for displaying urine flow information to a user. The smart device includes a transceiver for receiving data from a uroflowmetry device that detects a urine level in the uroflowmetry device and a number of rotations of a paddle wheel that rotates in response to urine exiting the uroflowmetry device. The received data includes a urine flowrate based on both the urine level and the number of rotations of the paddle wheel. Also included is a display and a processor. The processor is configured to, while the user is urinating, display the urine flowrate on the display via a software application to provide real-time urination feedback to the user.

### BRIEF DESCRIPTION OF THE FIGURES

FIG. 1 is a schematic view of a patient's urinary tract.

FIG. 2A is a schematic view of a feedback data plot for normal uroflow, according to an aspect of the disclosure.

FIG. 2B is a schematic view of a feedback data plot for dysfunctional uroflow, according to an aspect of the disclosure.

FIG. 2C is a schematic view of two feedback data plots that show correction of uroflow performance as a result of biofeedback therapy, according to an aspect of the disclosure.

FIG. 3 is a view of a network diagram showing communication between a uroflowmetry device and other devices of the uroflow system, according to an aspect of the disclosure.

FIG. 4A is a perspective view of a uroflowmetry device attached to a toilet, according to an aspect of the disclosure.

FIG. 4B is another perspective view of the uroflowmetry device in FIG. 4A unattached to the toilet, according to an aspect of the disclosure.

FIG. 4C is another perspective view of the uroflowmetry device in FIG. 4B with an insert pan removed for clarity, according to an aspect of the disclosure.

FIG. 5 is an exploded perspective view of a meter device of the uroflowmetry device, according to an aspect of the disclosure.

FIG. 6A is a perspective view of a pan insert of the meter device in FIG. 5, according to an aspect of the disclosure.

FIG. 6B is a perspective view of a funnel of the meter device in FIG. 5, according to an aspect of the disclosure.

FIG. 6C is a perspective view of a baffle of the meter device in FIG. 5, according to an aspect of the disclosure.

FIG. 6D is another perspective view of the funnel of the meter device in FIG. 5 showing placement of a pressure sensor, according to an aspect of the disclosure.

FIG. 6E is an inverted perspective view of the funnel in FIG. 6B, according to an aspect of the disclosure.

FIG. 6F is a perspective view of a cover of the meter device in FIG. 5, according to an aspect of the disclosure.

FIG. 6G is a perspective view of the cover of the meter device attached to the bottom of the funnel in FIG. 6B and including an attached paddle wheel, according to an aspect of the disclosure.

FIG. 7 is a side schematic view of the meter device in FIG. 6G, according to an aspect of the disclosure.

FIG. 8 is a flowchart of a user interface for controlling uroflowmetry session information displayed to the patient, according to an aspect of the disclosure.

FIG. 9A is a view of a user interface for selecting an operational mode of the uroflowmetry device, according to an aspect of the disclosure.

FIG. 9B is a view of a user interface showing uroflowmetry in fact mode, according to an aspect of the disclosure.

FIG. 9C is a view of a user interface showing uroflowmetry in fun mode, according to an aspect of the disclosure.

FIG. 9D is another view of a user interface showing uroflowmetryin fun mode, according to an aspect of the disclosure.

FIG. 10 is a flowchart showing operation of the uroflowmetrysystem, according to an aspect of the disclosure.

### DETAILED DESCRIPTION

In the following detailed description, numerous specific details are set forth by way of examples in order to provide a thorough understanding of the relevant teachings. However, it should be apparent to those skilled in the art that the present teachings may be practiced without such details. In other instances, well known methods, procedures, components, and circuitry have been described at a relatively high-level, without detail, in order to avoid unnecessarily obscuring aspects of the present teachings.

### Introduction

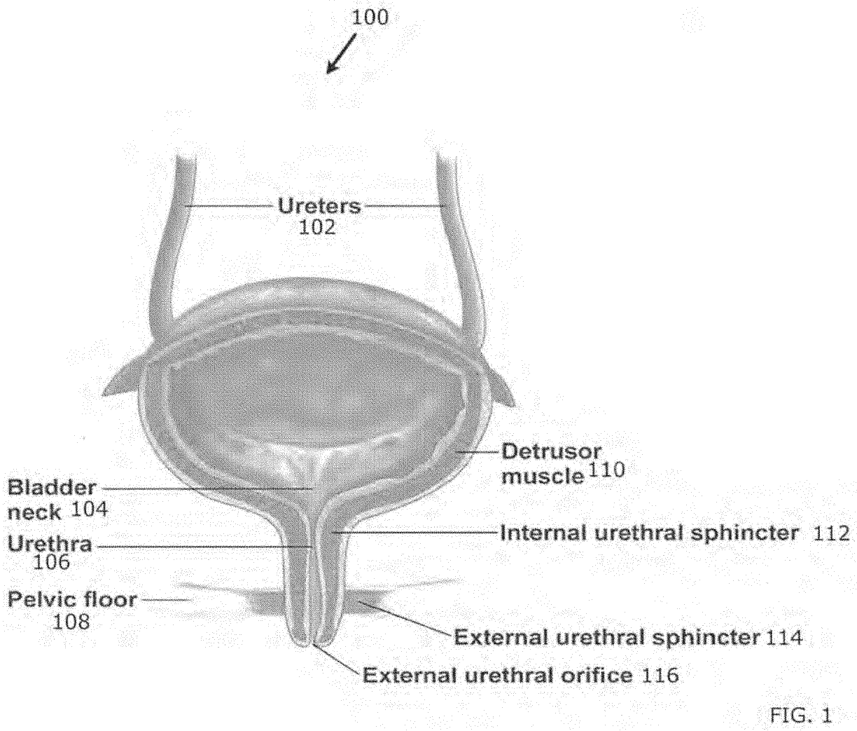

FIG. 1 is a view of the urinary tract 100 of a human. The upper urinary tract includes the kidneys (not in figure) and the ureters 102, while the lower urinary tract (LUT) includes the bladder neck 104, urethra 106, pelvic floor 108, detrusor muscle 110, internal urethral sphincter 112, external urethral sphincter 114 and external urethral orifice 116.

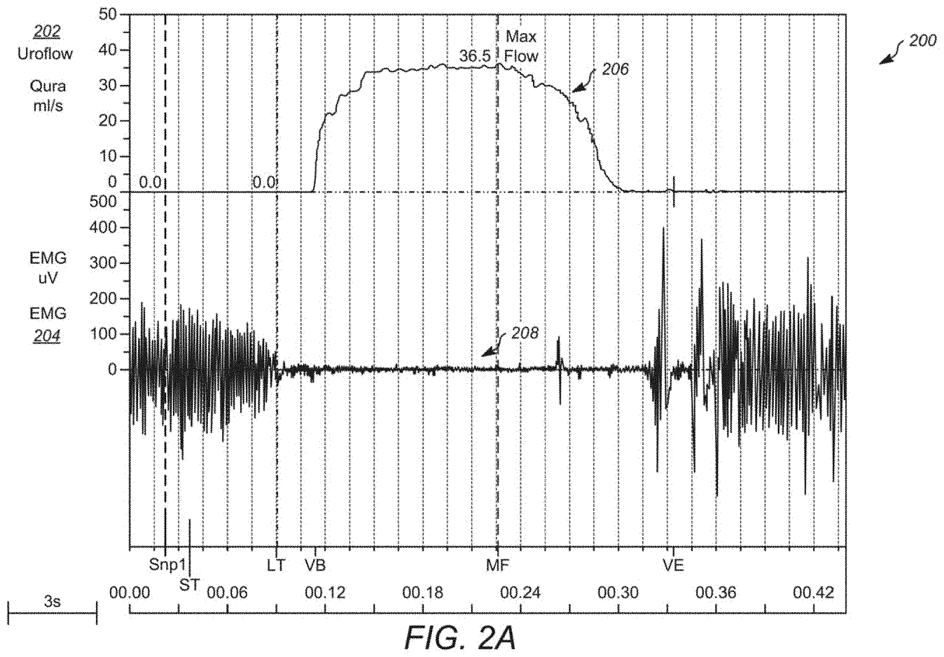

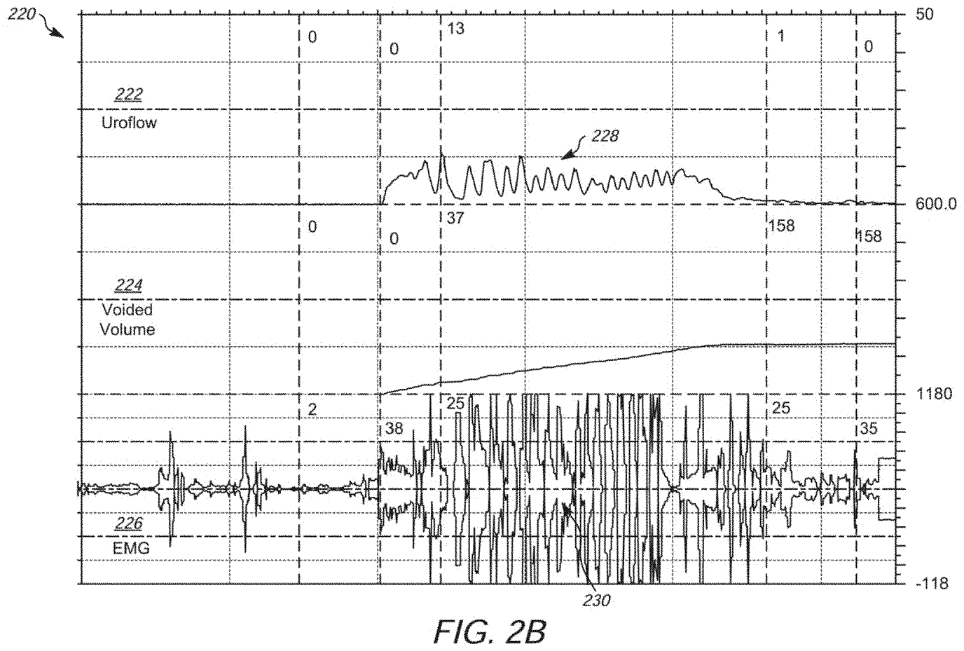

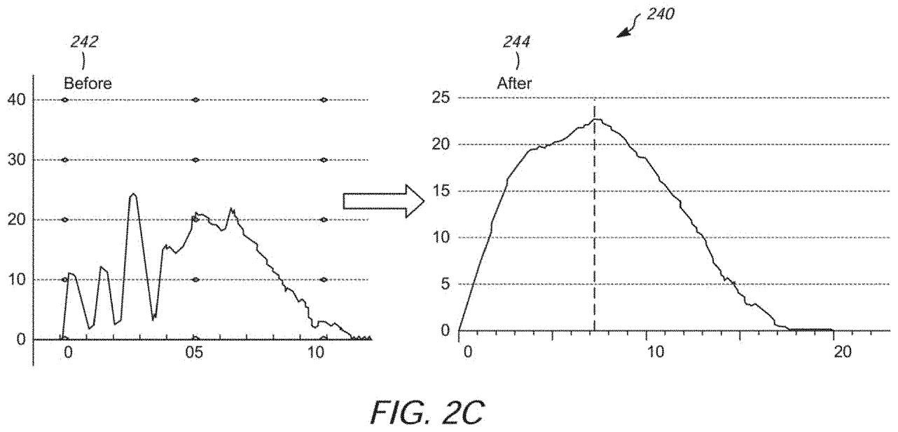

Normal voiding of the bladder is caused in by coordination between relaxation of the urethral sphincter and contraction of the detrusor muscle. A normal functioning LUT therefore results in bell-shaped uroflow pattern when measured by a uroflowmetry device. An example of measurements 200 in FIG. 2A show a normal voiding where uroflow pattern 202 and electromyography (EMG) 204 of the sphincter muscles are plotted versus time. As shown, the uroflow 202 has a bell-shaped curve 206, while the EMG 204 includes a flat portion 208 during voiding (e.g. the sphincter is relaxed).

LUT dysfunction, however, affect a significant portion of children. LUT dysfunction manifests in urinary holding maneuvers, urinary incontinence, urinary urgency, urinary frequency and urinary retention. Common causes of LUT dysfunction include dysfunctional voiding of the bladder, overactive bladder, voiding postponement and primary bladder neck dysfunction.

Dysfunctional voiding of the bladder is caused in part by habitual contractions of the urethral sphincter during voiding, which occurs in otherwise neurologically normal children. These habitual contractions result in increased resistance to outflow which often results in a staccato uroflow pattern when measured by a uroflowmetry device. An example of measurements 220 in FIG. 2B show dysfunctional voiding where uroflow pattern 222 and EMG 226 are plotted versus time. As shown, the plotted uroflow 222 has a staccato curve 228, while the EMG 226 includes an erratic section 230 during voiding. This results in a slow and possibly incomplete voiding of the bladder as shown in voided volume curve 224.

Biofeedback therapy provides the patient with dysfunctional voiding with a visual measurement or representation of their uroflow performance during uroflow sessions. For example, the patient can visually see the uroflow data curves in FIGS. 2A and 2B. Based on this visual, the patient can attempt to adjust control of their pelvic floor muscles to improve uroflow performance. For example, as shown in plots 240 of FIG. 2C a patient may have a dysfunctional uroflow pattern 242 before biofeedback therapy and a more normal uroflow pattern 244 after numerous biofeedback sessions. Biofeedback allows the user to learn how to better control their muscles to attain a more normal uroflow pattern.

Due to the benefits of biofeedback for treating LUT dysfunction, especially dysfunctional voiding, Applicant has developed a uroflowmetry device that can be mounted to a standard toilet seat (e.g. in a patient's home). The uroflowmetry device is self-draining into the toilet and provides real-time biofeedback in the form of a data graph or an animation, for example an animated game, to the patient's wireless device (e.g. smartphone). This uroflowmetry device makes it easier, cleaner and more comfortable for the patient (especially a child) to practice controlling their LUT muscles on a daily basis in their home. The uroflow device is also compatible with both male and female patients.

### System Communication

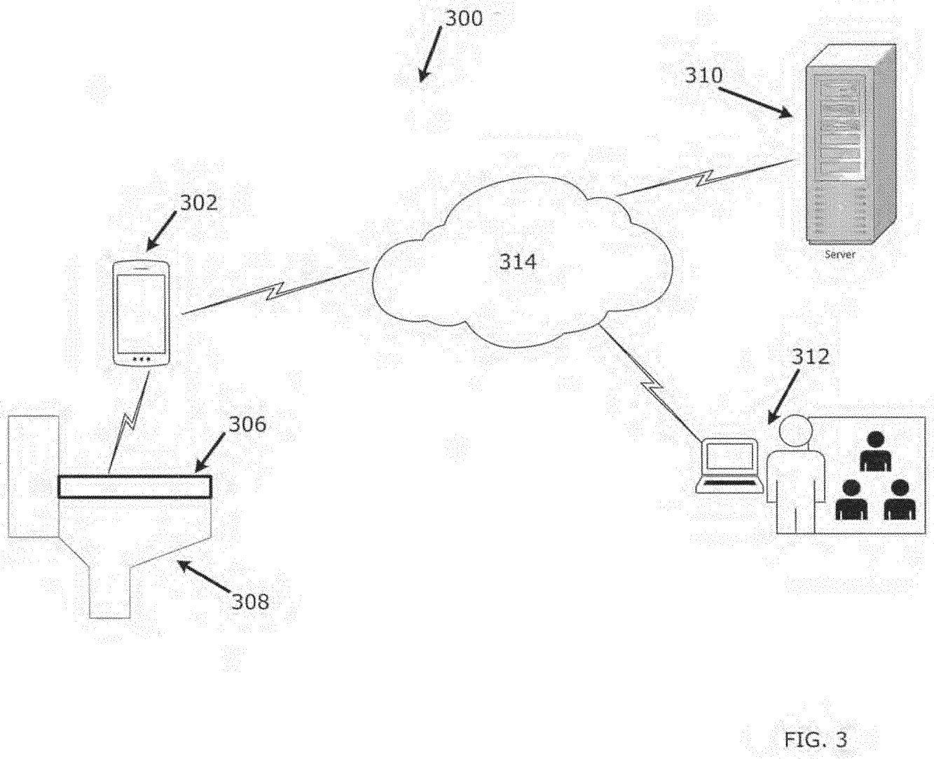

The general operation of the uroflowmetry system 300 will now be described with reference to FIG. 3. As shown in FIG. 3, uroflowmetry device 306 is mounted to the seat of toilet 308. The details of the mounting hardware/procedure will be described later. Uroflowmetry device 306 communicates wired or wirelessly (e.g. Bluetooth) to a patient device 302 (e.g. smartphone, tablet, laptop, etc.) which displays real-time feedback (e.g. data graph, animation, audio/visual instructions, etc.) to the patient during the uroflowmetry session. This real-time feedback may be based on urine flowrate of the patient, urine volume over the session, or any other measurable/computable quantity related to urination.

In addition, patient device 302 may upload the results of the uroflowmetry session to server 310 via network 314 (e.g. Internet). A medical professional 312 (e.g. physician, nurse, etc.) may then download these results from server 310 via network 314 and interpret the results. This allows the medical professional to monitor the patient progress. In addition, the medical professional may communicate with patient device 302 via network 314 to adjust the biofeedback accordingly (e.g. change the type of visual/audio feedback in an attempt to aid in patient progress).

### Device Hardware

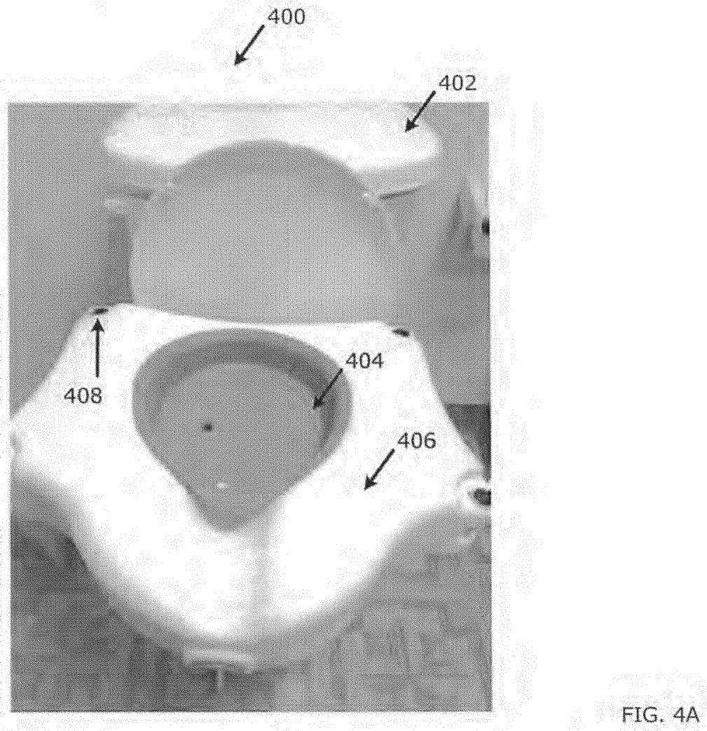

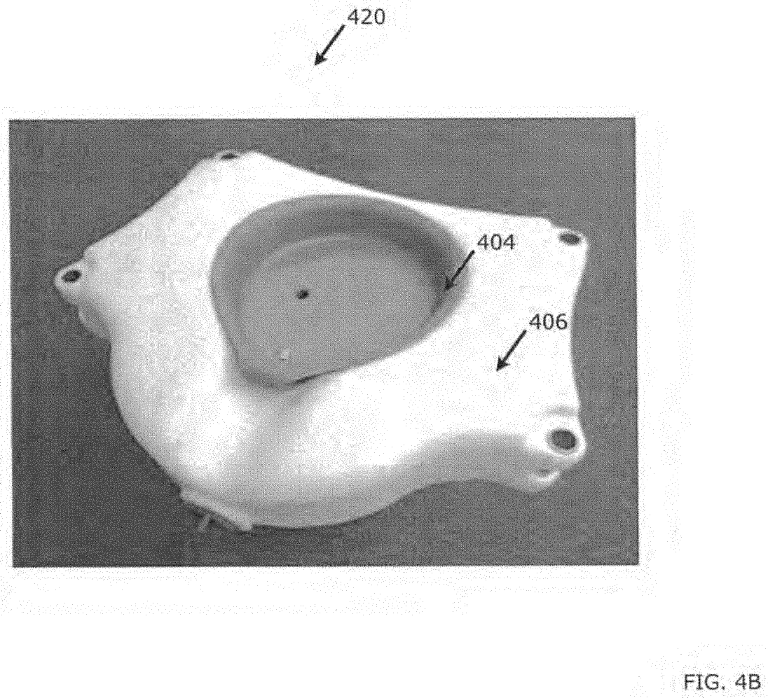

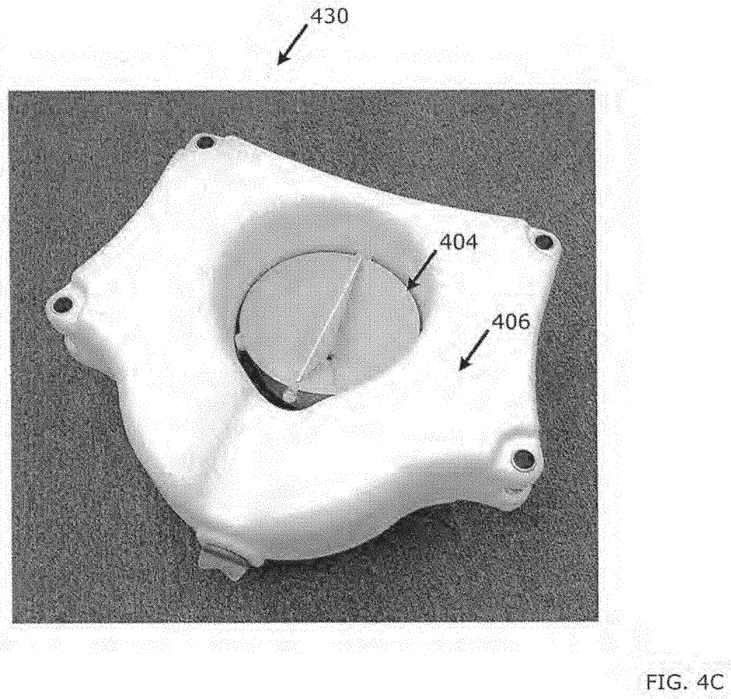

The uroflowmetry device in FIG. 3 is now shown in more detail as view 400 in FIG. 4A, where a uroflowmetry device is mounted to toilet 402. The uroflowmetry device generally includes a meter device 404 and a seat device 406 which mounts to toilet 402 via clamps, suction cups, or the like. Mounting holes 408 may also be provided for mounting the seat device 406 to the toilet. Meter device 404, however, rests inside an opening of seat device 406. When using the uroflowmetry device during a uroflow session, a patient can urinate into meter device 404 from either a sitting position (e.g. sitting on seat device 406) or a standing position. For clarity, another view of the uroflowmetry device is shown in FIG. 4B where uroflowmetry device 420 is not mounted to the toilet.

FIG. 4C is a perspective view of uroflowmetry device 420 with an insert pan removed to show further internal details of meter device 404. As will be described in detail later, meter device 404 includes a funnel and a baffle. In general, when the patient urinates into meter device 404, the insert pan (see FIG. 4B), the funnel and the baffle (see FIG. 4C) guide the urine into the toilet while measuring the uroflow metrics such as flowrate and volume.

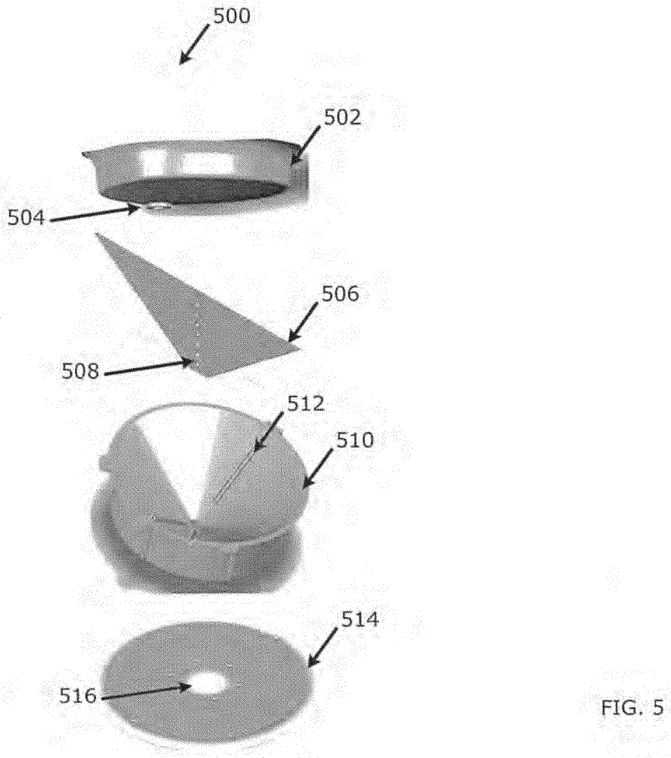

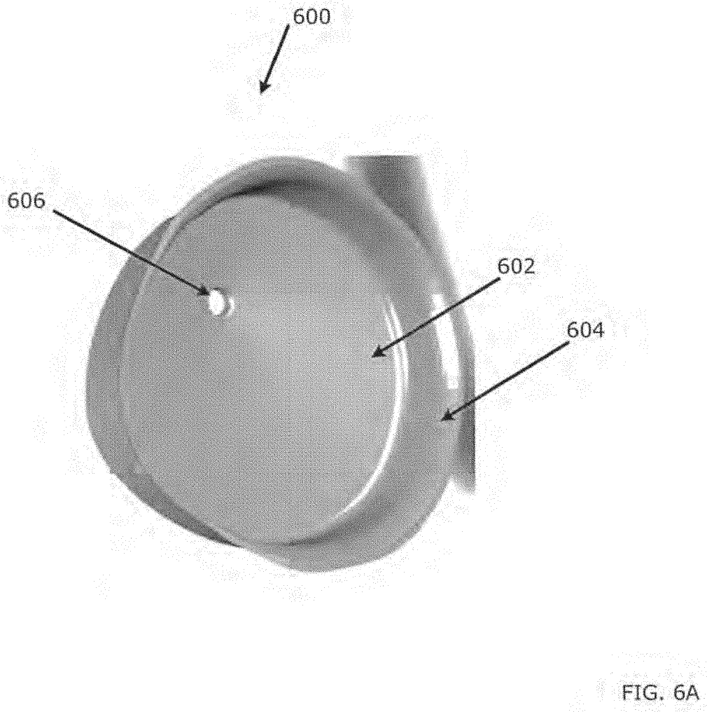

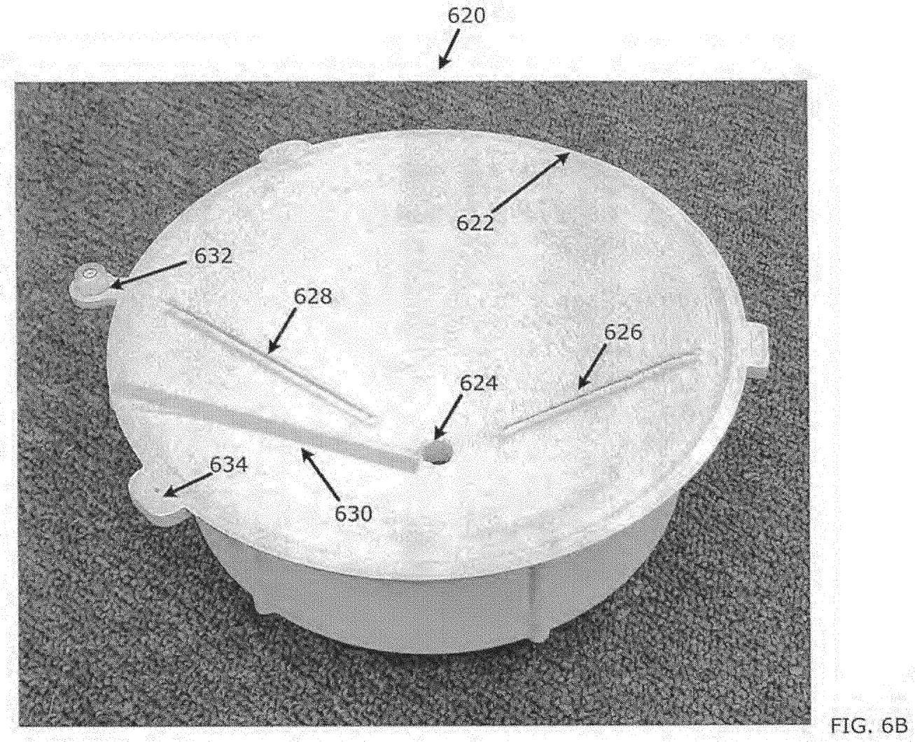

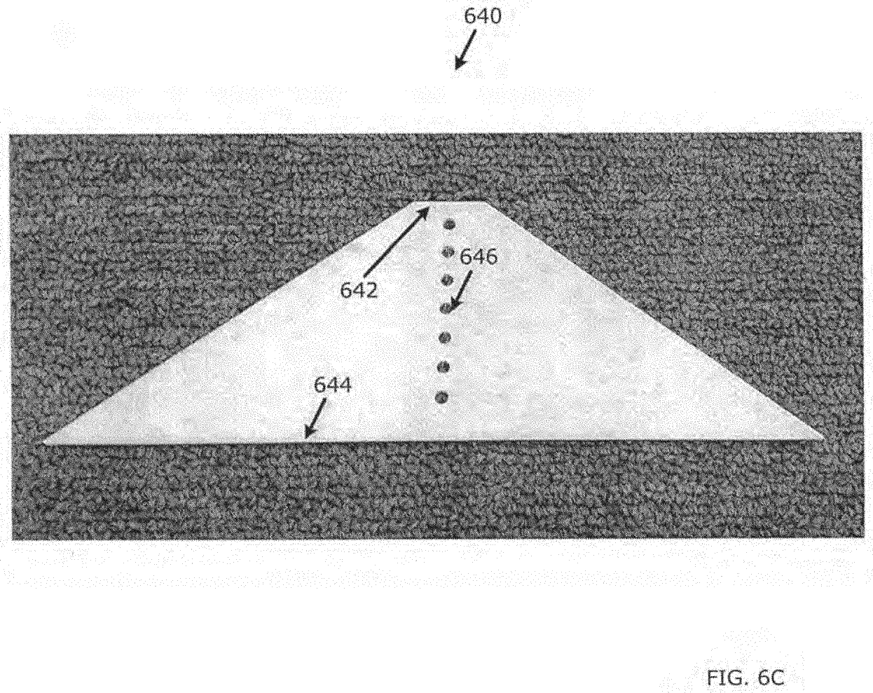

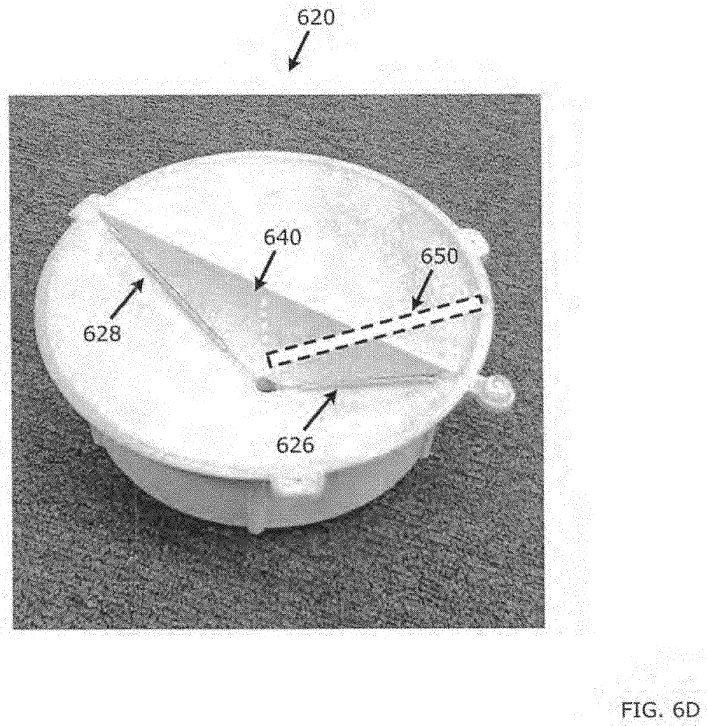

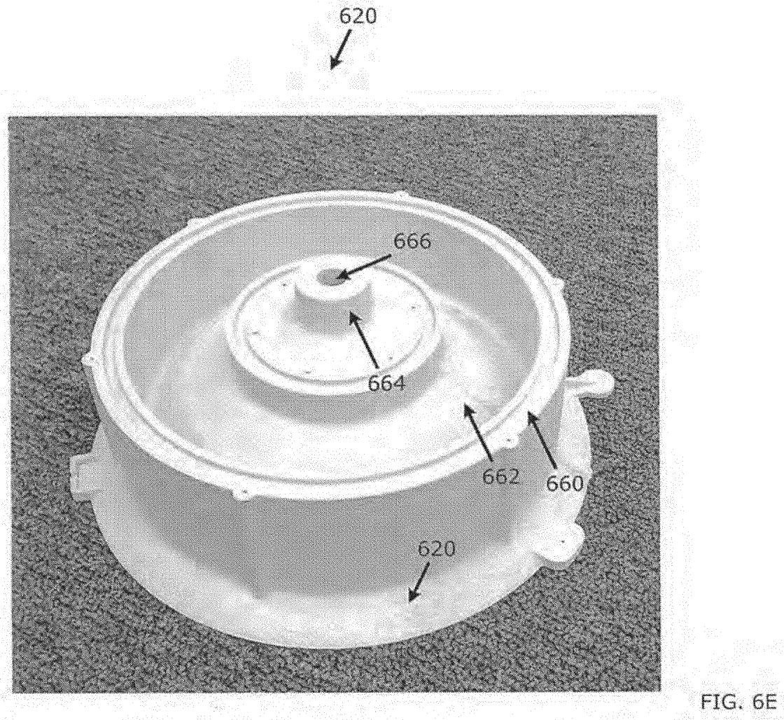

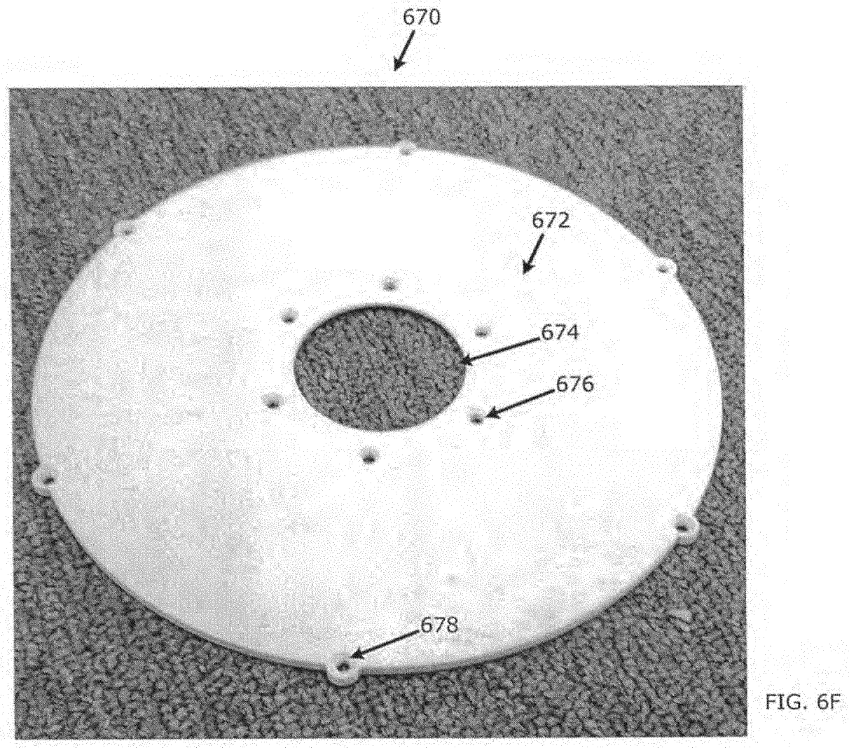

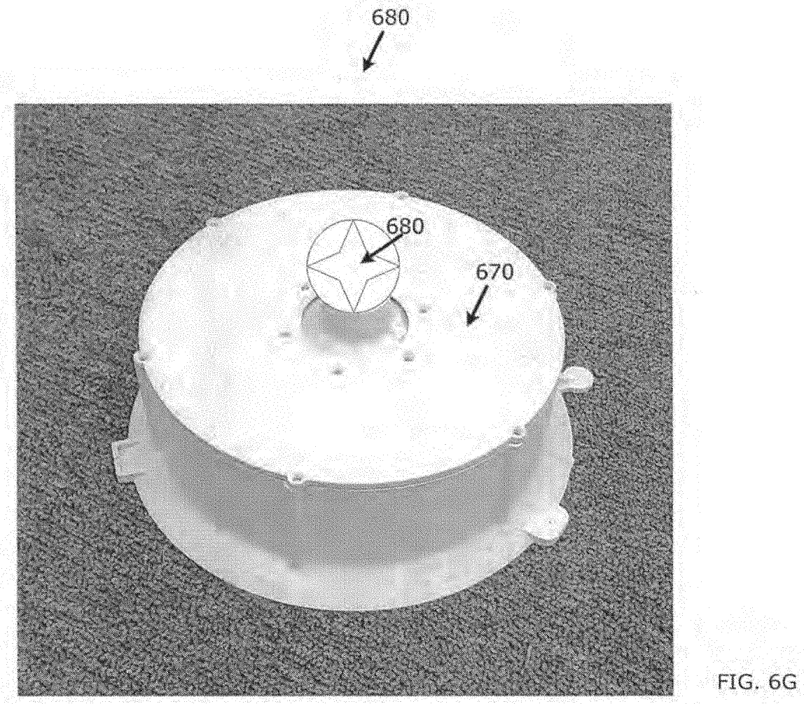

FIG. 5 is an exploded structural view 500 of the meter device 404 shown in FIGS. 4A-4C. Specifically, meter device 404 includes insert pan 502 having hole 504, baffle 506 having holes 508, funnel 510 having grooves 512 and cover 514 having hole 516. Each of these structural components will now be described in detail with respect to FIGS. 6A-6G.

FIG. 6A is a perspective view 600 of insert pan 502. Insert pan 502 generally includes base 602, wall 604 and hole 606. Insert pan 502 collects urine directly from the patient's urine stream and channels the urine through hole 606 into an opening of funnel 510. This channeling action reduces turbulence in the urine that enters the funnel to achieve a more accurate measurement of uroflow metrics.

FIG. 6B is a perspective view 620 of funnel 510 which includes a wide top portion and a narrow bottom portion to channel the urine entering the funnel from top rim 622 down to draining hole 624. In addition, funnel 510 also includes channels 626/628 to hold baffle 506 in place and channel 630 to hold a sensor (not shown) in place. Furthermore, funnel 510 includes wings 634 for seating/mounting funnel 510 into the opening of seat device 406. Bubble level 632 may also be provided to ensure that funnel 510 is properly leveled when mounted. Although not shown, set screws or the like may be inserted into wings 634 to provide level mounting to seat device 406.

FIG. 6C is a perspective view 640 of baffle 506. Baffle 506 has a substantially triangular shape that extends from a narrow bottom portion 642 to a wider top portion 644. The shape of baffle 506 is set to fit the internal shape of funnel 510. This shape allows baffle 506 to be seated in channels 512 (e.g. channels 626/628 in FIG. 6B) of funnel 510. Baffle 506 may be permanently or temporarily installed (e.g. removable) in channels 626/628 of funnel 510. Also, as shown in FIG. 6C, baffle 506 may also include holes 646 for allowing urine to flow between different sides of the baffle. However, holes 646 are not necessary. When installed in funnel 510, baffle 506 has the function of reducing turbulence of urine flowing into funnel 510. This ensures that urine levels rise within the funnel in a controlled manner.

FIG. 6D is another perspective view 620 of funnel 510 showing placement of a sensor 650. Specifically, a sensor 650 is installed permanently (e.g. glued in) or temporarily (e.g. snapped in) in channel 630 of funnel 510. Sensor 650 may extend from the upper rim 622 of the funnel down to the opening of the funnel draining hole 624. In one example, sensor 650 is a pressure sensor. Pressure sensor 650 may be an air tube connected to a diaphragm (not shown). As weight of the urine accumulated in the funnel presses on the air tube, the air pressure deforms the air tube and the diaphragm. The amount of deformation of the diaphragm generally corresponds to the height of the urine level in the funnel. In general, as more urine is present in the funnel a greater amount of deformation will occur in the diaphragm due to the pressure. The deformation amount is then output as an electrical signal which can be used to determine urine volume within the funnel. It should be noted, however, that sensor 650 does not have to be a pressure sensor. Sensor 650 can be any type of sensor (e.g. resistive, capacitive, etc.) to measure the amount of urine accumulated in funnel 510.

FIG. 6E is an upside-down perspective view 620 of the funnel shown, for example, in FIG. 6B. As shown in FIG. 6E, the funnel includes a round wall 660 that extends from flange 620 of the funnel towards the draining hole of the funnel resulting in empty cavity 662. Also included is extension 664 that extends funnel draining hole 624 to an opening 666 beyond round wall 660. In general, the urine collected by the funnel exits the funnel via opening 666.

FIG. 6F is a perspective view 670 of a cover 672 for covering cavity 662 shown in FIG. 6E. Cover 672 includes opening 674 and mounting holes 676/678 on the inner/outer diameter of the cover. As shown in view 680 of FIG. 6G, the cover 672 is attached to the bottom (e.g. to wall 660) of the funnel. Although not shown, cover 672 can be attached with mounting hardware (e.g. screws) via holes 676/678. Also shown is a paddle wheel 680 mounted to opening 666 of the funnel. As will be described later, paddle wheel 680 spins as urine exits opening 666 of the funnel draining hole. The number of rotations (e.g. the number of complete 360° revolutions or partial revolutions) of paddle wheel 680 may be used to compute uroflow metrics (e.g. urine flowrate of the patient).

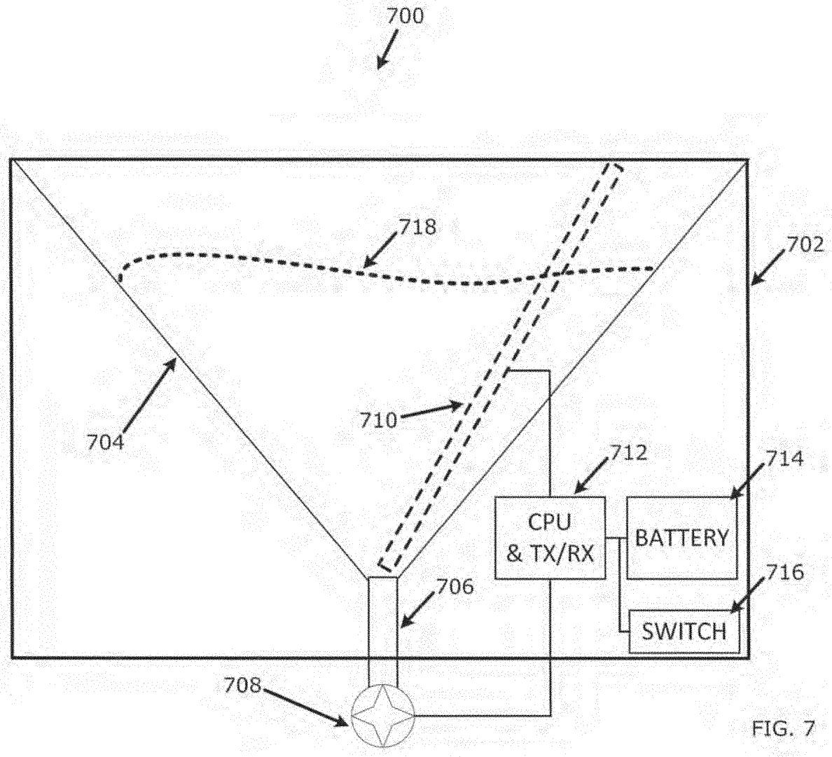

The operation of meter device 404 will now be described with reference to FIG. 7 which shows a simplified side schematic view 700 of meter device 404. Meter device 404 includes outer round wall 702, funnel 704, funnel tube 706, paddle wheel 708, sensor 710, controller 712, battery 714 and switch 716 (baffle not shown for clarity). During a uroflowmetry session, the patient urinates into the top opening of funnel 704. The urine accumulates to reach a urine level 718 in the funnel (assuming a higher volume of urine is being input to the funnel opening than can be output through the funnel drain hole). The urine exiting through the funnel drain hole saturates paddle wheel 708, which begins to spin at a rotational speed according to the speed of urine flowing through the funnel drain hole. This urine speed is generally based on the height of the urine level in the funnel (e.g. the higher the urine level, the faster the paddle wheel spins due to the increased pressure).

During operation, optional switch 716 allows power to flow from battery 714 to controller 712, sensor 710 and paddle wheel 708. Switch 716 may be a mechanical switch, magnetic switch or the like that is actuated by the patient, or actuated when the meter device 404 is inserted into seat device 406. Upon power-up (e.g. when switch 716 is closed), controller 712 receives electrical measurement signals from sensor 710 (e.g. pressure signal from a pressure sensor) and a number of rotational pulse signals from paddle wheel 708. For example, paddle wheel 708 may output a magnetic pulse with each full or partial rotation. The pressure signal and the paddle wheel pulses are then used by a central processing unit (CPU) in controller 712 to compute uroflow metrics such as urine flowrate of the patient which is then transmitted (e.g. Bluetooth or the like) by a transceiver (TX/RX) in controller 712 to patient device 302 (e.g. smartphone).

For example, the CPU breaks down the uroflowmetry session into measurement periods (e.g. 100 ms) during which urine flowrate of the patient is computed and then transmitted to the smartphone in real time for plotting. These measurement periods are repeated throughout the duration of the uroflowmetry session.

During each measurement period, the CPU determines a first urine level in the funnel based on the pressure signal measured at a first time, and a second urine level in the funnel based on the pressure signal measured at a second time. The first and second urine level correspond to a respective first and second urine volume based on the known geometry of the funnel. The CPU then determines a rate of change in funnel urine volume. For example, the rate of change in funnel urine volume may be computed (e.g. in real-time or prior to operation and stored in a table), by dividing the difference between the first urine volume and the second urine volume by the difference between the first time and second time.

During operation, if there is large urine flowrate into the funnel (e.g. more than the exiting flow of the paddle wheel), the pressure sensor will show a positive change in pressure and therefore positive change (i.e. rate) in volume during the measurement period. If, there is a small urine flowrate into the funnel (e.g. less than the exiting flow of the paddle wheel), the pressure sensor will show a slight negative change in pressure and therefore a slight negative change in volume during the measurement period. If there is no flow into the funnel, the pressure sensor will show a negative change that is equal to the rate of change in urine volume exiting the funnel as determined by the paddle wheel sensor, and the summation of the two rates will be zero.

During each measurement period, the CPU also determines a number of paddle wheel rotations (e.g. complete or partial rotations) by counting the paddle wheel pulses between a first time and a second time. The number of paddle wheel rotations correspond to a rate of change in urine volume exiting the funnel (e.g. Vol/sec) based on the known geometry of the paddle wheel (e.g. each revolution of the paddle wheel corresponds to a known volume of urine exiting the funnel).

The CPU then determines the urine flowrate of the patient based on both the rate of change in funnel urine volume and the rate of change in urine volume exiting the funnel. For example, the CPU may add the rate change in funnel urine volume to the rate of change in urine volume exiting the funnel in order to compute the urine flowrate of the patient during each measurement period. The urine flowrate of the patient during each measurement period may then be integrated for the duration of the session and transmitted to the smartphone for display to the patient.

In another embodiment, the raw pressure signals and the rotational pulses may be transmitted by controller 712 via TX/RX to patient device 302 which then computes the uroflow metrics (e.g. urine flowrate of the patient, urine volume, etc.). In either scenario, the uroflow metrics such as urine flowrate of the patient and urine volume are displayed to the patient via a data plot, numerical output or an animation such as an interactive game.

### Software Application

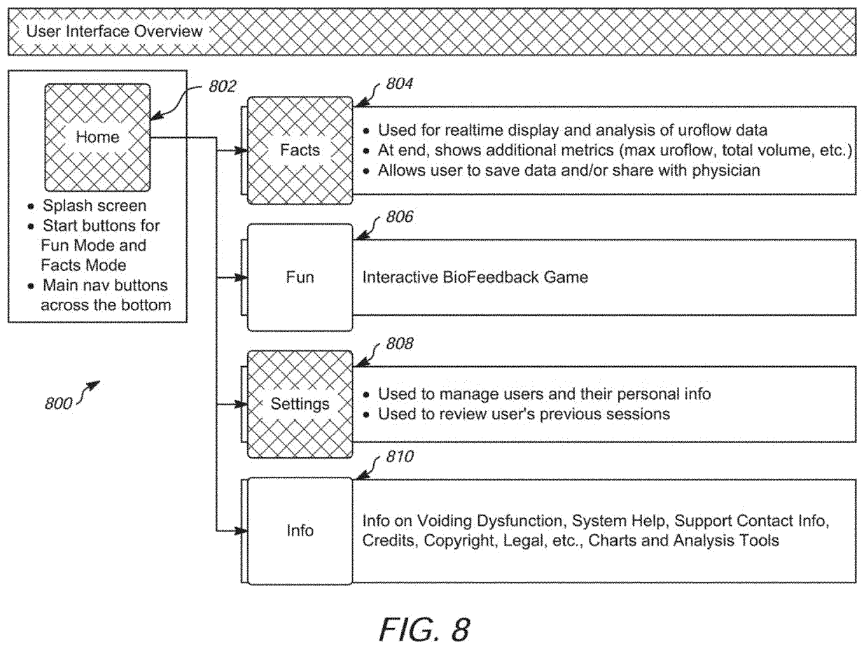

As described above, uroflow metrics such as urine flowrate of the patient and urine volume are displayed to the patient to provide real-time feedback during uroflowmetry sessions. FIG. 8 is an example flowchart 800 of a user interface for controlling how the feedback is displayed to the patient during the uroflowmetry session. In one example, home screen 802 may have various buttons such as mode selection buttons and navigation buttons. This allows the patient to select facts mode 804 used for real-time display and analysis of uroflow metrics, interactive game 806 used for real-time interaction with the patient in a biofeedback manner, settings 808 to manage patient personal information and review previous uroflowmetry sessions, and information 810 such as information on voiding dysfunction, system help, support contact, etc. Other options may also be available and selectable by the patient.

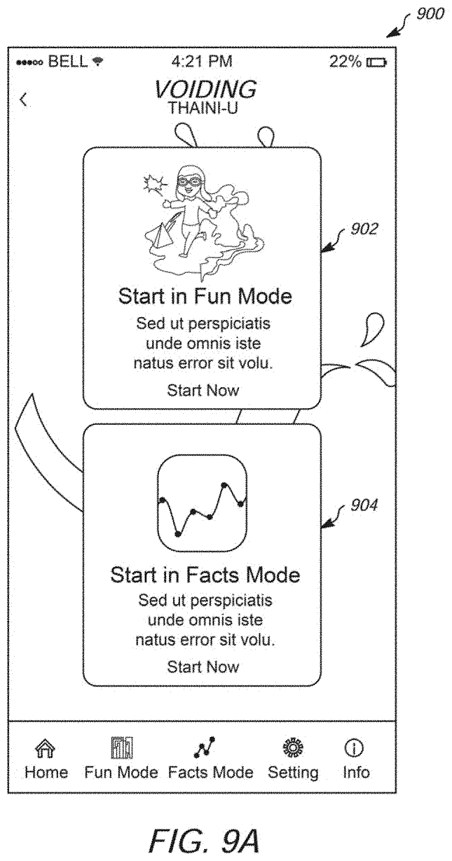

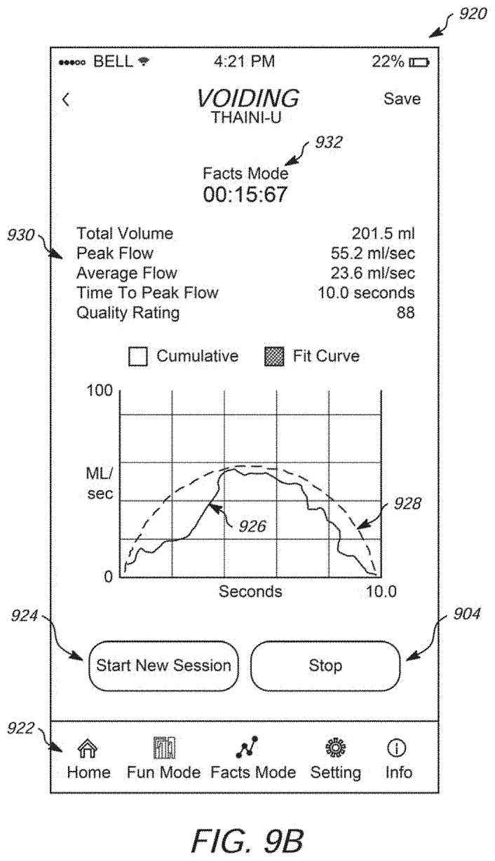

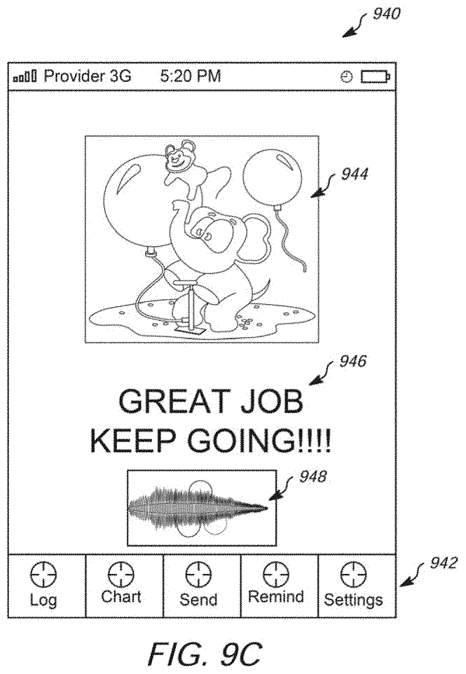

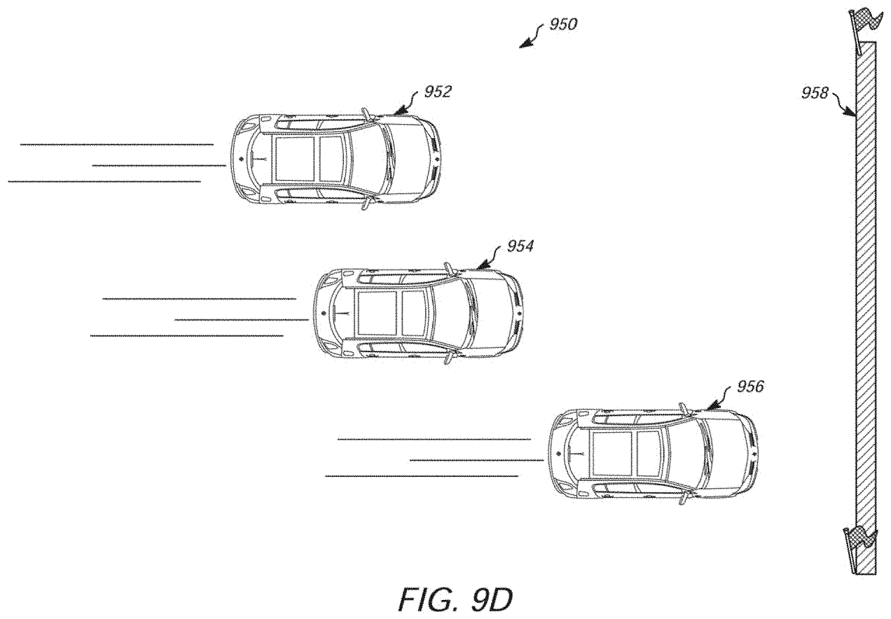

An example of a home screen 802 is shown as screen 900 in FIG. 9A. In this home screen, the software application displays options for selecting a fun (e.g. interactive game for biofeedback) mode 902 and facts (e.g. data plots, etc.) mode 904, as well as navigation buttons (e.g. home, mode selections, settings and other info)

If the patient, for example, selects facts mode 902, they may be presented with the screen 920 shown in FIG. 9B which displays urine timing data 932, uroflow metrics 930 and data plots such as urine flowrate 926 and fit curve 928. Also included are control buttons 924 for starting/stopping uroflowmetry session and navigation buttons 922. In one example, to start a uroflowmetry session, the patient can press the start new session button 924 and then begin urinating. Once finished, the patient can press the stop session button. In another example, the start/stop buttons are not needed, and the application will start/stop analysis/transmission automatically based receiving uroflow data detected by meter device 404.

In another example, if the patient selects fun mode 904, they may be presented with the screen 940 shown in FIG. 9C which displays an animation 944 of a character performing a task such as blowing up balloon, encouraging (e.g. coaching) comments 946 as well as audio output 948. For example, if the patient is able to generate more normal urine flowrate, the balloon begins to expand, with the ultimate goal of popping the balloon. Therefore, the more normal the patient's urine flowrate becomes, the better performance they achieve in the animation. This type of biofeedback is especially helpful for children patients.

In yet another example, if the patient selects fun mode 904, they may be presented with the screen 950 shown in FIG. 9D which displays an animated game such as cars 952, 954 and 956 racing towards finish line 958. As the patient increases their urine flowrate to normal levels, their designated car (e.g. car 956) picks up more speed, with the ultimate goal of winning the race. Therefore, the more normal the patient's urine flowrate becomes, the better performance they achieve in the game.

### Operation Example

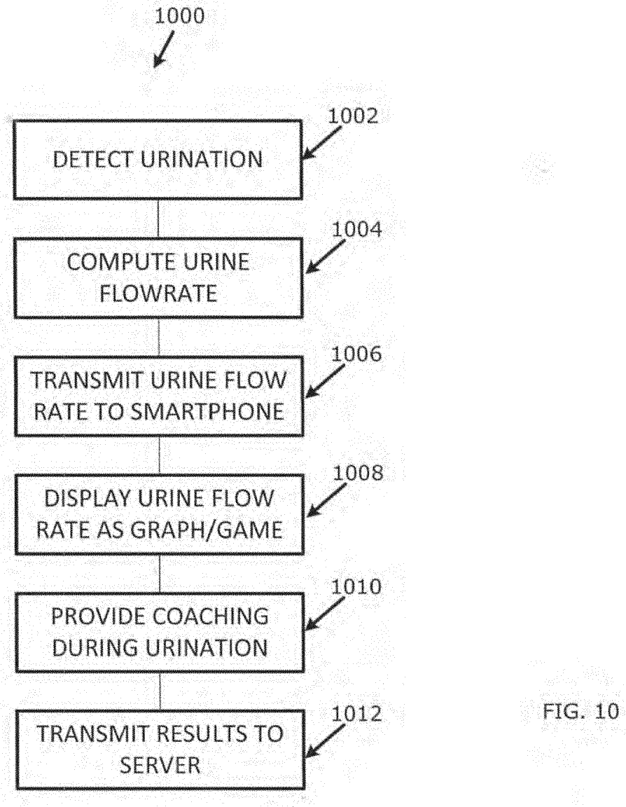

FIG. 10 is a flowchart 1000 showing an example operation of the uroflowmetry system. In step 1002, meter device 404 (already mounted in seat 406) determines urine flowrate based on the detection of the pressure sensor 710 at two points in time and the number of rotations of paddle wheel 708 between the two points in time. In step 1004 the patient's urine flowrate is computed by controller 712 (e.g. by determining the rate of change in funnel urine volume and the rate of change in urine volume exiting the funnel, and adding the two values together), and then transmitted by controller 712 (e.g. via Bluetooth transceiver) to patient's personal device 302 in step 1006. Alternatively, the patient's urine flowrate may be computed by patient's personal device 302 upon receiving the pressure data and paddle wheel rotational data from meter device 404.

In either scenario, once the patient's urine flowrate and possibly other uroflow metrics are computed, they are displayed in step 1008 on the patient's personal device 302 in real-time (e.g. during the session). As described previously, uroflow metrics may be displayed in a standard data type format (e.g. numbers/graphs, etc.) or in a fun type format (e.g. animation, games, etc.). In addition, in step 1010 text or audio commands (e.g. coaching information) may also be output to the patient to help guide the patient to achieve an improved result during urination (i.e., biofeedback). This coaching information may include encouraging messages or instructional messages. Once the uroflowmetry session is complete, patient's personal device 302 transmits (e.g. via Wi-Fi, cellular or the like) the results in step 1012 to the server. A medical professional can then review the results stored on the server and make an assessment of the patient's current state and progress.

### Conclusion

The steps in FIGS. 8-10 may be performed by the controller 712 in FIG. 7 and/or the server in FIG. 3, upon loading and executing software code or instructions which are tangibly stored on a tangible computer readable medium, such as on a magnetic medium, e.g., a computer hard drive, an optical medium, e.g., an optical disc, solid-state memory, e.g., flash memory, or other storage media known in the art. In one example, data are encrypted when written to memory, which is beneficial for use in any setting where privacy concerns such as protected health information is concerned. Any of the functionality performed by the computer described herein, such as the steps in FIGS. 8-10 may be implemented in software code or instructions which are tangibly stored on a tangible computer readable medium. Upon loading and executing such software code or instructions by the computer, the controller may perform any of the functionality of the computer described herein, including the steps in FIGS. 8-10 described herein.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” “includes,” “including,” or any other variation thereof, are intended to cover a non-exclusive inclusion, such that a process, method, article, or apparatus that comprises or includes a list of elements or steps does not include only those elements or steps but may include other elements or steps not expressly listed or inherent to such process, method, article, or apparatus. An element preceded by “a” or “an” does not, without further constraints, preclude the existence of additional identical elements in the process, method, article, or apparatus that comprises the element.

Unless otherwise stated, any and all measurements, values, ratings, positions, magnitudes, sizes, and other specifications that are set forth in this specification, including in the claims that follow, are approximate, not exact. Such amounts are intended to have a reasonable range that is consistent with the functions to which they relate and with what is customary in the art to which they pertain. For example, unless expressly stated otherwise, a parameter value or the like may vary by as much as ±10% from the stated amount.

In addition, in the foregoing Detailed Description, it can be seen that various features are grouped together in various examples for the purpose of streamlining the disclosure. This method of disclosure is not to be interpreted as reflecting an intention that the claimed examples require more features than are expressly recited in each claim. Rather, as the following claims reflect, the subject matter to be protected lies in less than all features of any single disclosed example. Thus, the following claims are hereby incorporated into the Detailed Description, with each claim standing on its own as a separately claimed subject matter.

While the foregoing has described what are considered to be the best mode and other examples, it is understood that various modifications may be made therein and that the subject matter disclosed herein may be implemented in various forms and examples, and that they may be applied in numerous applications, only some of which have been described herein. It is intended by the following claims to claim any and all modifications and variations that fall within the true scope of the present concepts.

1. A uroflowmetry device comprising:
a funnel for funneling urine of a user;
a sensor for measuring urine level in the funnel over a measurement period;
a paddle wheel operable to rotate in response to urine exiting the funnel and generate a signal after each rotation during said measurement period;
a transmitter for transmitting data from the uroflowmetry device to a smart device; and
a processor configured to, while the user is urinating:
compute a urine flowrate based on urine level in the funnel and a number of rotations of the paddle wheel over the measurement period, and
transmit the urine flowrate to the smart device to provide real-time urination feedback to the user.

2. The uroflowmetry device of claim 1, further comprising:
an insert pan positioned at the opening of the funnel, the insert pan funneling the urine into the funnel opening.

3. The uroflowmetry device of claim 1, further comprising:
a baffle positioned inside an opening of the funnel to reduce urine turbulence, the baffle having a substantially triangular shape.

4. The uroflowmetry device of claim 1,
wherein the sensor is a pressure sensor that detects pressure caused by the urine level in the funnel.

5. The uroflowmetry device of claim 1, further comprising:
a bubble level for leveling the uroflowmetry device.

6. The uroflowmetry device of claim 1, further comprising:
a mounting structure for mounting the uroflowmetry device to a toilet, wherein the funnel is seated in an opening of the mounting structure.

7. The uroflowmetry device of claim 1, further comprising:
a switch that is activated when the funnel is seated in the mounting structure; and
a battery for powering the processor and the transmitter,
wherein the switch connects the processor to the battery, or instructs the processor to begin computing and transmitting the urine flowrate to the smart device.

8. The uroflowmetry device of claim 1,
wherein the processor is configured to compute the urine flowrate by:
determining a rate of change in funnel urine volume based on a change in the urine level in the funnel during the measurement period,
determining a rate of change in urine volume exiting the funnel based on the number of rotations of the paddle wheel during the measurement period, and
adding the rate of change in funnel urine volume to the rate of change in urine volume exiting the funnel to calculate the urine flowrate.

9. The uroflowmetry device of claim 1,
wherein the funnel includes surface ridges to reduce urine turbulence.

10. The uroflowmetry device of claim 1,
wherein the mounting structure includes clamps for mounting the funnel to the toilet seat.

11. A smart device for displaying urine flow information to a user, the smart device comprising:
a transceiver for receiving data from a uroflowmetry device that detects a urine level in the uroflow device and a number of rotations of a paddle wheel that rotates in response to urine exiting the uroflowmetry device, the received data including a urine flowrate based on both the urine level and the number of rotations of the paddle wheel;
a display; and
a processor configured to, while the user is urinating, display the urine flowrate on the display via a software application to provide real-time urination feedback to the user.

12. The smart device of claim 11,
wherein the processor is further configured to display the urine flowrate on the display as a data graph.

13. The smart device of claim 11,
wherein the processor is further configured to display the urine flowrate on the display as an interactive game with which the user interacts.

14. The smart device of claim 11,
wherein the processor is further configured to display coaching advice on the display instructing the user on proper techniques for normal uroflow.

15. The smart device of claim 11,
wherein the processor is further configured to transmit, via the transceiver, the urine flowrate to a server, the urine flowrate being accessible via the server by medical professionals.

16. The smart device of claim 11,
wherein the a transceiver is configured to receive, from the uroflowmetry device, the urine level in the uroflow device and the number of rotations of the paddle wheel, and
wherein the processor is further configured to compute the urine flowrate based on the urine level in the uroflowmetry device and the number of rotations of the paddle wheel.

17. The smart device of claim 11,
wherein the processor is further configured to control the display to display control buttons, allowing the user to control computation of urine flowrate and a display format for the urine flowrate.

18. The smart device of claim 11,
wherein the processor is further configured to control the display to display historical urine flowrate data to the user.

19. The smart device of claim 11,
wherein the transceiver is paired with the uroflowmetry device via Bluetooth.

20. The smart device of claim 11,
wherein the processor is further configured to compute and display a best-fit curve based on the urine flowrate.
